(19) United States
(12) Patent Application Publication
(10) Pub. No.: US 2010/0057216 A1
(43) Pub. Date: Mar. 4, 2010

(54) System and method for joint resurfacing with dynamic fixation

(76) Inventors: Jamy Gannoe, West Milford, NJ (US); Jeff Tyber, Bethlehem, PA (US)

Correspondence Address: WARD & OLIVO, SUITE 300, 382 SPRINGFIELD AVENUE, SUMMIT, NJ, 07901, US

(21) Appl. No.: 12/460,493

(22) Filed: Jul. 20, 2009

Related U.S. Application Data
(60) Provisional application No. 61/135,715, filed on Jul. 23, 2008.

(51) Int. Cl. A61F 2/42; A61B 17/86; A61B 17/56

(52) U.S. Cl. 623/21.18; 606/301; 606/86 R

(57) ABSTRACT

A joint resurfacing system for repairing defects in the articular surface of bones in the foot. The system includes a hemiarthroplasty implant for the talus resulting in minimal removal of the underlying bone. The implant device comprises a first curved surface and a second curved surface, with the second curved surface coupled to an articular surface of a talus bone via a dynamic fixation device.

### CROSS-REFERENCE TO RELATED APPLICATION

This application claims the benefit of Provisional Application No. 61/135,715, filed Jul. 23, 2008, the entire contents of which are herein incorporated by reference.

### FIELD OF THE INVENTION

This invention relates to the field of joint prosthesis, and more particularly, to a joint resurfacing device and system for repairing defects in the articular surface of bones in the foot.

### BACKGROUND OF THE INVENTION

The talus is located in the center of the ankle and is primarily responsible for load transfer from the tibia through the entire foot. The talus is one of the main load bearing articulating surfaces within the human ankle and is commonly the location of ankle pain. Ankle pain is commonly associated with the presence of osteochondral lesion and joint arthritis. There are several methods of relieving ankle pain that range from non-surgical approaches to arthroscopic and surgical procedures. One cause of ankle pain is the location of osteochondral lesions on the medial and lateral talar dome. The talar dome is a common location for lesions surpassed only by the knee and possibly the elbow. The formation of lesions has been attributed to several factors but the location of these lesions on the talar dome is primarily attributed to the talar dome equator bearing the maximum stress during load bearing activities. A common method of correcting these deficiencies in the knee is by performing arthroplasty, arthrodesis or a hemiarthroplasty.

In addition to osteochondral lesions, arthritis of the ankle is another common pathology effecting ankle pain. Ankle arthritis is commonly treated by a wide variety of surgical procedures, ranging from steroid injections, arthrodesis and total joint replacement. With the exception of injections and medications, the arthrodesis and total joint replacement procedures commonly require six weeks in a non-weight bearing cast, six weeks in a walking cast and rehabilitation in addition to the extensive recovery time. However, these aforementioned procedures commonly reduce the natural ankle kinematics.

Hemiarthroplasty is a common procedure performed within the human knee. Generally, a hemiarthroplasty results from only a portion of the native articulating surface being damaged and requiring a partial arthroplasty procedure. This results in a proportional section of the damaged tissue being removed and replaced with an implant serving as the new articulating surface, while leaving intact most of the native cartilage as possible. However, this is currently limited within the human ankle due to the amount of bone that is removed to make space for the implant. In addition, the means of fixation has been well documented as an issue with keeping the implant fixed to the bone where migration and settling issues have been prevalent.

Currently, there are only a few implants designed to aid in the correction of osteocondral lesions and severe ankle arthritis without performing arthroplasty or arthrodesis. While talus resurfacing is known, resurfacing has been primarily done through an arthroscopic process without an implant. This arthroscopic process is limited to removal of large defects and replaced with autograph or allograph cartilage taken from a donor site.

Also, ankle fusion and total joint replacement are aggressive procedures with difficult revision options and are therefore typically reserved for late stage disease. In the ankle, there are few, if any, options for early surgical treatment that preserves the option for fusion, partial and total joint replacement, should they become necessary at a later time.

There is therefore a need for a joint resurfacing device and system and method of use that overcomes some or all of the previously delineated drawbacks of prior joint resurfacing devices.

### SUMMARY OF THE INVENTION

An object of the present invention is to overcome the drawbacks of previous inventions.

Another object of the present invention is to provide a novel and useful joint resurfacing device for restoring the articulating surface of the talus through a minimally invasive procedure.

Another object of the present invention is to restore the articulating surface through minimal distraction of the joint.

Another object of the present invention is to provide a system for restoring the articulating surface of a talus bone with a dynamic fixation assembly.

Another object of the present invention is to provide a method for resurfacing the articulating surface of the talus.

Another object of the invention is to provide an option for early, tissue sparing treatment, which preserves the option for subsequent revision surgery including fusion, partial and total joint replacement

Another object of the invention is to provide a means by which the ankle can be repaired via resurfacing the talar surface and then, in a modular manner, adding the tibial surface at a later date, if necessary.

In a first non-limiting aspect of the present invention, an implant device is provided for a talar joint resurfacing. The implant comprises a generally curved member having a lateral portion, a distal portion, an exterior surface and an inferior surface. The exterior surface is provided to be slidably coupled to a tibial joint. The inferior surface is provided to be coupled to a talar dome.

In a second non-limiting aspect of the present invention, a system for resurfacing a joint in a foot is provided. The system comprises an implant device for replacing damaged tissue at the talus, a trial inserter assembly and a guide assembly. The implant device includes a generally curved member having a lateral portion and a distal portion. The implant device has an exterior surface provided to be slidably coupled to a tibial joint. The implant also comprises an inferior surface selectively coupled to a talar dome in a threaded fixation device, where the threaded fixation device is selected from the group consisting of a dynamic fixation device or a static screw device.

In a third non-limiting aspect of the present invention, a system for resurfacing a joint in a foot is provided. The system comprises an implant device for replacing damaged tissue at the talus, a trial inserter assembly and a guide assembly. The implant device includes a generally curved member having a lateral portion and a distal portion. The implant device has an exterior surface provided to be slidably coupled to a tibial joint. The implant also comprises an inferior surface having an osteoconductive material, thereby causing the implant device to be selectively coupled to a talar dome.

In a fourth non-limiting aspect of the present invention, a system for resurfacing a joint in a foot is provided. The system comprises an implant device for replacing damaged tissue at the talus, a trial inserter assembly and a guide assembly. The implant device includes a generally curved member having a lateral portion and a distal portion. The implant device has an exterior surface provided to be slidably coupled to a tibial joint. The implant also comprises an inferior surface having fins or spikes, thereby causing the implant device to be selectively coupled to a talar dome.

In a fifth non-limiting aspect of the present invention, a method for resurfacing a joint in a foot is provided. First, a mid-foot incision is made in the foot. The talus in then prepared for restoration by distracting the tibial-talar joint and the damaged tissue is removed from the surface of the talus. Next, a trial inserter assembly is used to assist the surgeon in achieving the proper depth and alignment of implant device. After the correct trial-sized device has been determined, the surgeon will position the trial-sized device onto the talus and selectively couple guide assembly to the trial-sized device for accurate positioning of a drill guide. A guide wire is then installed and a hole is drilled in the talus using the guide wire. Following, an implant inserter is used to thread into the implant device. Once the implant device is installed, the threaded rod is pulled back through the tunnel until the implant device comes into contact with the talus. Next, the implant inserter is removed and a dynamic fixation device is inserted for ridged connection to the implant device using a screwdriver.

### BRIEF DESCRIPTION OF THE DRAWINGS

A further understanding of the present invention can be obtained by reference to a preferred embodiment set forth in the illustrations of the accompanying drawings. Although the illustrated embodiment is merely exemplary of systems and methods for carrying out the present invention, both the organization and method of operation of the invention, in general, together with further objectives and advantages thereof, may be more easily understood by reference to the drawings and the following description. The drawings are not intended to limit the scope of this invention, which is set forth with particularity in the claims as appended or as subsequently amended, but merely to clarify and exemplify the invention.

For a more complete understanding of the present invention, reference is now made to the following drawings in which:

FIG. 1 is a perspective view of an implant device used in the joint resurfacing system in accordance with a preferred embodiment of the present invention.

FIG. 2 is another perspective view of the implant device used in the joint resurfacing system in accordance with the preferred embodiment of the present invention.

FIG. 3 is a perspective view of an implant device used in the joint resurfacing system shown in FIGS. 1 and 2 according to the preferred embodiment of the present invention.

FIG. 4 is a front view of the implant device shown in FIG. 3 according to the preferred embodiment of the invention.

FIG. 5 is a right sided view of the implant device shown in FIG. 3 according to the preferred embodiment of the invention.

FIG. 6 is a bottom view of the implant device shown in FIG. 3 according to the preferred embodiment of the invention.

FIG. 7 is a perspective view of the trial inserter assembly of the joint resurfacing system according to the preferred embodiment of the present invention.

FIG. 8 is a perspective view of the guide assembly of the joint resurfacing system according to the preferred embodiment of the present invention.

FIG. 9A illustrates a step of installing the implant device of FIG. 3 using the trial inserter assembly of FIG. 7 according to the preferred embodiment of the present invention.

FIG. 9B illustrates another step of installing the implant device of FIG. 3 using the trial inserter assembly of FIG. 7 and the guide assembly of FIG. 8 according to the preferred embodiment of the present invention.

FIG. 9C illustrates another step of installing the implant device of FIG. 3 using a trial inserter according to the preferred embodiment of the present invention.

FIG. 9D illustrates another step of installing the implant device of FIG. 3 using a screwdriver according to the preferred embodiment of the present invention.

FIG. 10 is a flow chart illustrating the method of coupling the implant device shown in FIGS. 1-9D to talus bone in a patient's foot according to the preferred embodiment of the present invention.

### DETAILED DESCRIPTION

The present invention may be understood more readily by reference to the following detailed description of preferred embodiment of the invention. However, techniques, systems and operating structures in accordance with the present invention may be embodied in a wide variety of forms and modes, some of which may be quite different from those in the disclosed embodiment. Consequently, the specific structural and functional details disclosed herein are merely representative, yet in that regard, they are deemed to afford the best embodiment for purposes of disclosure and to provide a basis for the claims herein, which define the scope of the present invention. It must be noted that, as used in the specification and the appended claims, the singular forms “a”, “an”, and “the” include plural referents unless the context clearly indicates otherwise.

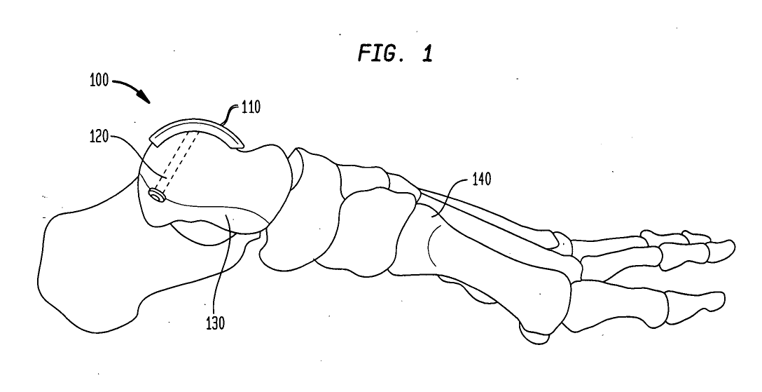

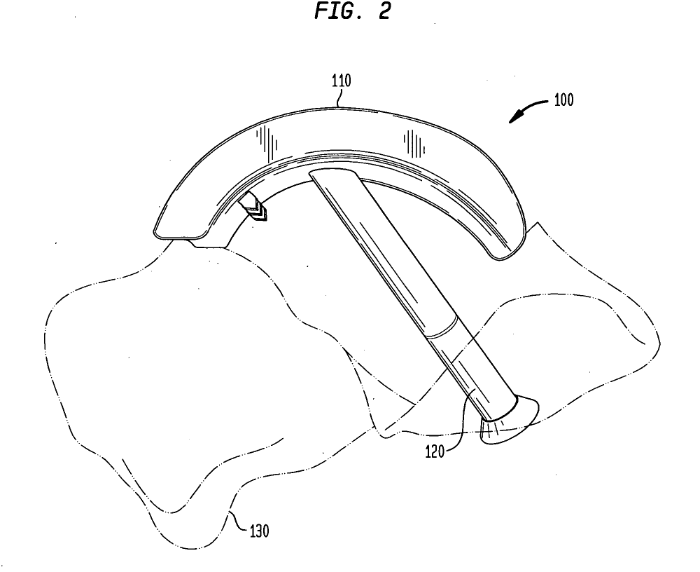

Referring now to FIGS. 1 and 2, there is shown a joint resurfacing system 100 in accordance with the teachings of the preferred embodiment of the invention. As shown, joint resurfacing system 100 includes an implant 110 selectively attached to a talus bone 130 of a human foot 140 in order to replace any deteriorated or damaged bone or tissue. A dynamic fixation device 120 is selectively coupled to implant 110 for applying a dynamic compressive force on implant 110, thereby securely coupling implant 110 to the talus 130. It should be appreciated that implant 110 is used to perform hemiarthroplasty using a minimal invasive procedure for restoration of the articulating surface of the talus 130. In other non-limiting embodiments, implant 110 may resurface only part of talus 130. As such, implant 110 may be cut along its width to conform to the portion of talus 130 that is being replaced.

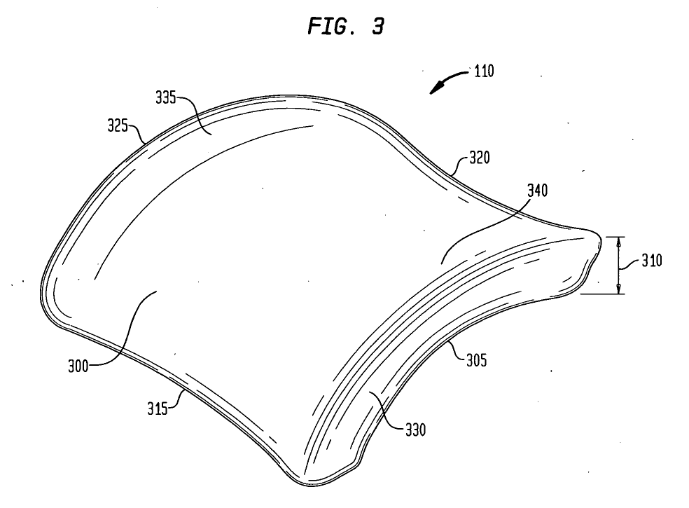

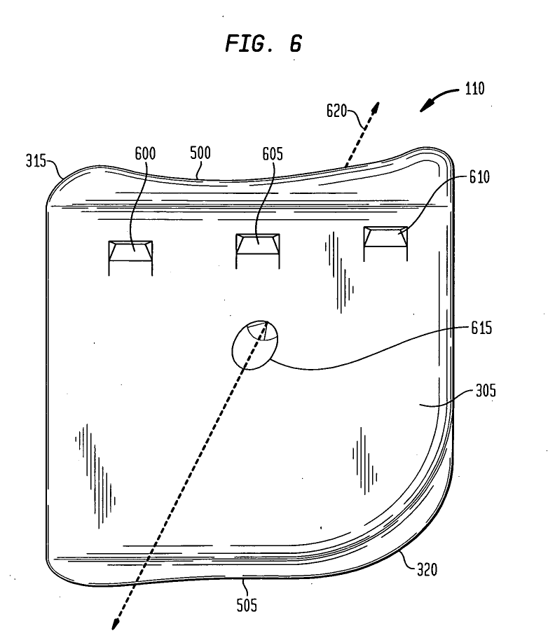

As shown in FIGS. 3, 4, 5 and 6, implant 110 is generally curved in shape (i.e., implant 110 has a generally “potato-chip” shape). As shown in FIG. 3, implant 110 has a lateral portion 335 and a medial portion 340 that cooperatively form the body of implant 110. Implant 110 has a first superior surface 300 and an opposed inferior surface 305 (which will be shown in FIG. 6). Further, implant 110 has a first anterior edge 315, an opposed posterior second edge 320, a left edge 325 and a right edge 330. Also, implant 110 has a thickness 310, preferably comprising a range of 0.5-15 mm.

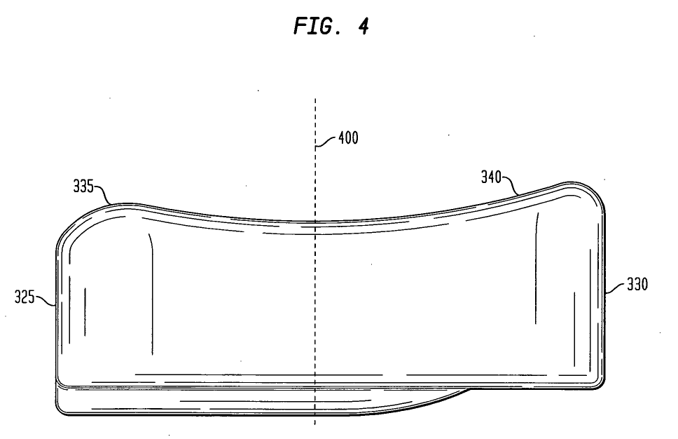

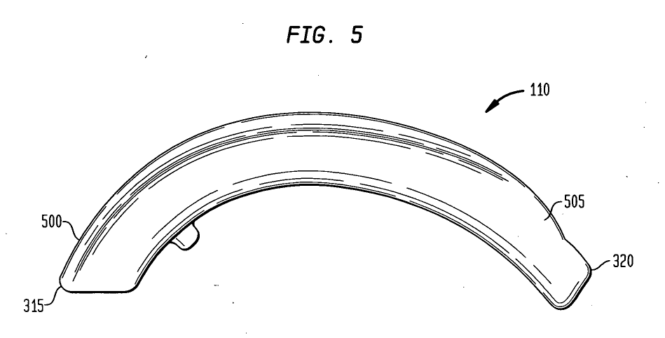

As shown in FIG. 4, implant 110 is designed with several distinct radii to mimic the native tissue as well as the tomography of the native healthy talus and distal tibia connection. The radii consist of medial convex portion 340 and lateral convex portion 335 that meet in the approximate equator 400 in a concaved manner. As shown in FIG. 5, implant 110 has a gradual increasing radius having the smallest radii located at the anterior position 500 and the posterior position 505.

Further, as shown in FIG. 6, implant 110 has a plurality of substantially similar holding spikes 600, 605 and 610 formed on inferior surface 305. Holding spikes 600, 605 and 610 each have a tapered edge provided to cause holding spikes 600, 605 and 610 to travel through bone material as implant 110 couples to the bone surface. As such, holding spikes 600, 605 and 610 provide additional primary stability to implant 110. Implant 110 also comprises a threaded aperture or hole 615 provided on surface 305. Hole 615 has a plurality of substantially similar internal threads (not shown) provided to receive a complementary threaded end of a dynamic fixation device 120 to secure implant 110 to talus bone 130. Alternatively, hole 615 comprises inner surface capable of being threaded by the threaded end of dynamic fixation device 120.

It should be appreciated that in one non-limiting embodiment, implant 110 may be made from a Titanium material, although, in other non-limiting embodiments, implant 110 may be made from Cobalt Chrome, Stainless Steel, PEEK, PE, PEEK, NiTi or any other biocompatible material. It should also be appreciated that thickness 310 of implant 110 (previously shown in FIG. 3) has beneficial properties, such as a lower bulk modulus (i.e., resistance to uniform compression), resulting in lower stress shielding while reducing the amount of removed native tissue. This reduction in removal of native tissue reduces the violation of the talar-tibial joint. It should also be appreciated that inferior surface 305 may be coated with a porous undercoating such as plasmacoated Titanium, mesh Titanium, or other similar types of osteoconductive materials, in order to provide an osteoconductive surface for fixating implant 110 to the talus surface. In other non-limiting embodiments, surface 305 may be infused with hydroxylapatite or other similar types of growth hormones to increase connection to the surface of the talus.

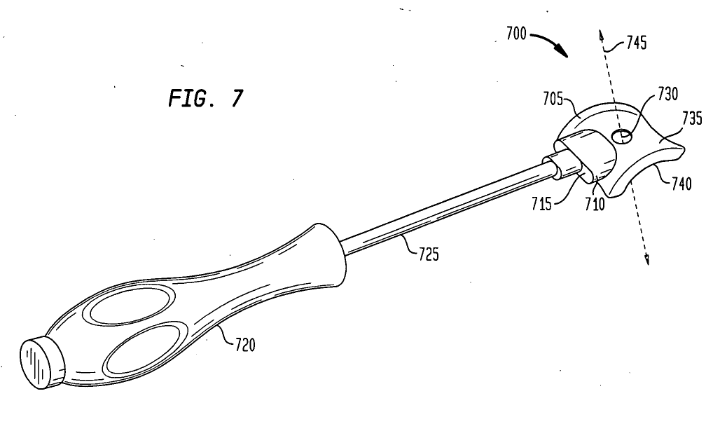

FIG. 7 illustrates a trial inserter assembly 700 for preparing talus 130 for insertion of implant 110. Particularly, trial inserter assembly 700 includes a trial device 705 that is substantially similar to implant device 110 as was shown in a previous embodiment in FIGS. 3-6. Trial-sized device 705 has a curved body and a rectangular shaped contact portion 710 provided with a groove 715 to receive rod portion 725. Rod portion 725 emanates from portion 710 and terminates into handle portion 720. Trial-sized device 705 further comprises aperture or hole 730, which traverses trial device 705 from top surface 730 to bottom surface 740. Aperture or hole 730 is aligned along axis 745, which is the same as axis 620 of implant device 110. Similarly, aperture or hole 730 is positioned at the same location as aperture or hole 615 of implant device 110. In operation, trial-sized device 705 would be utilized to assist the surgeon in achieving the proper depth, positioning and alignment of implant 110. Trial-sized device 705 also would assist the surgeon in estimating the size of an implant that is needed for talus resurfacing. Additionally, trial-sized device 705 would be utilized as an anchoring system for the drill guide, as will be later shown and described.

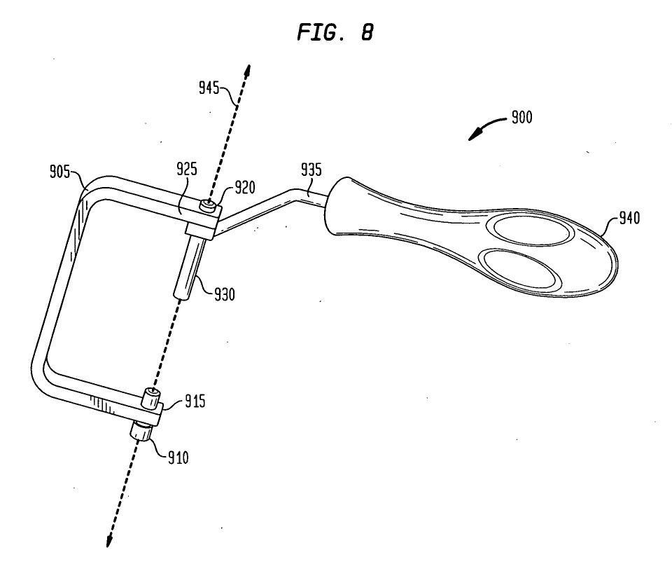

Guide assembly 900 is illustrated in FIG. 8. Guide assembly 900 is utilized for assisting in alignment of dynamic fixation device 120. Particularly, guide assembly 900 has a generally “U-shaped” portion 905 selectively coupled to a cannulated locating tube 910 at first end 915 and also to a cannulated guide wire tube 920 at second end 925. Guide assembly 900 further comprises drill guide rod 930 selectively coupled to second end 925. Handle 940 is coupled to drill guide rod 930 through a rod 935. Drill guide rod 930, guide wire tube 920 and locating tube 910 are all aligned along axis 945. In operation, guide assembly 900 is utilized an anchoring system for the drill guide (not shown) and guide wire, as well as to assist the surgeon in accurate positioning of the implant.

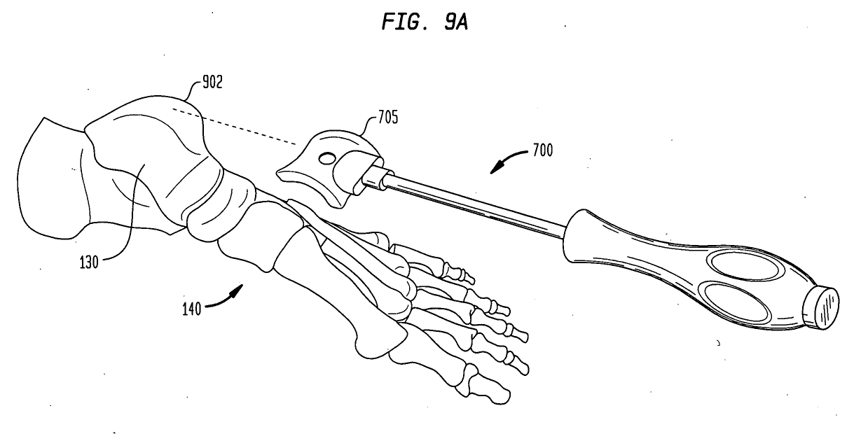

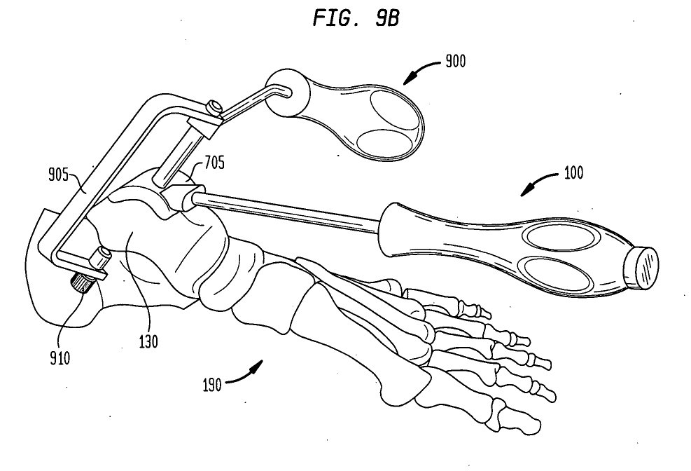

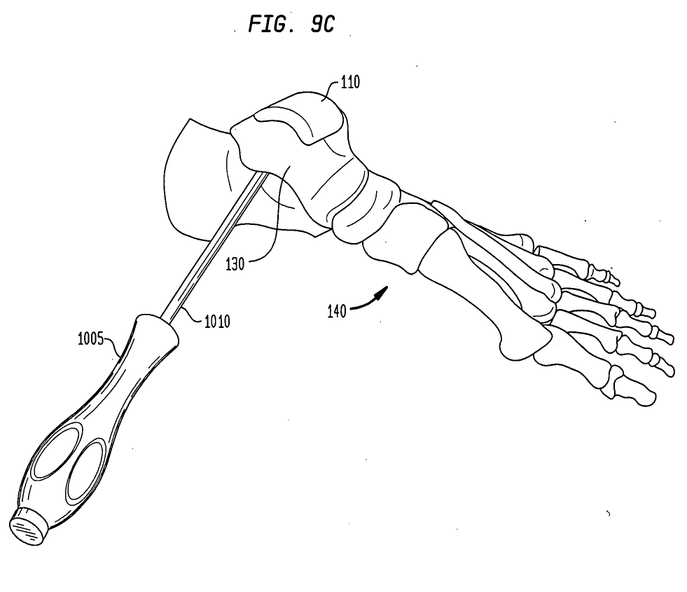

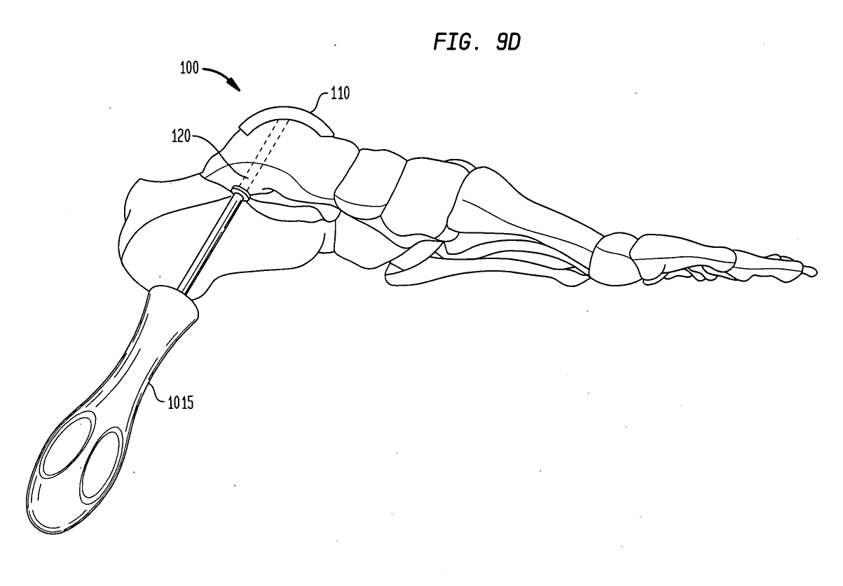

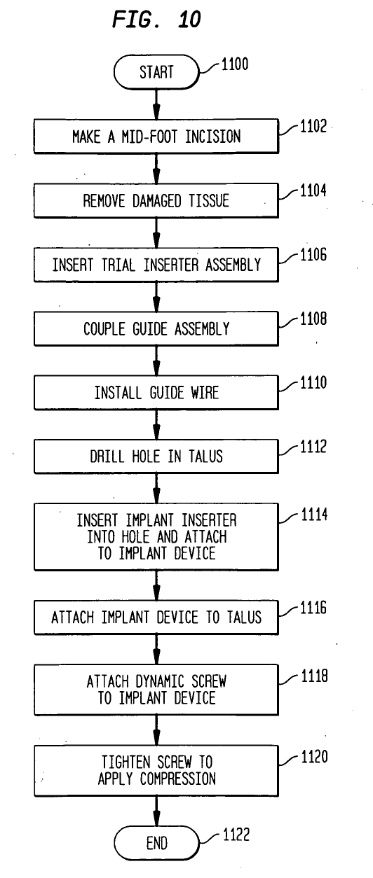

In operation and as best shown in FIGS. 9A-9D and 10, implant device 110, trial inserter assembly 700 and guide assembly 900 may be utilized to provide a system for restoration of articulating surface 902 of talus 130 in a human foot 140. The method starts in step 1100 and proceeds to step 1102, whereby a mid-foot incision is made in foot 140. Next, in step 1104, foot 140 is prepared for restoration by distracting (i.e., separating) the tibial-talar joint and removing the damaged tissue from surface 902. The removal of tissue may include the removal of at least the bone or tissue that has been deteriorated or damaged. Next, in step 1106, as shown in FIG. 9A, a trial inserter assembly 700 is used to assist the surgeon in achieving the proper depth and alignment of implant device 110. Trial inserter assembly 700, having a trial-sized device 705, is designed not only to allow for accurate positioning of implant device 110, but also doubles as the anchoring system for a drill guide (not shown). This allows the surgeon to, accurately, position a fixation hole for implant device 110 insertion and connection of implant device 110 to dynamic fixation device 120 (shown in FIG. 9C).

Next in step 1108, as shown in FIG. 9B, after the correct sized trial-sized device 705 has been determined, the surgeon will position trial-sized device 705 onto talus 130 and selectively couple guide assembly 900 to trial-sized device 705. Guide assembly 900 allows the surgeon to visualize the placement of the exiting guide wire (not shown), such as a Kirschner wire, through a posterior stab incision. Next, in step 1110, the placement of the guide wire (not shown) will allow a cannulated trial insert (not shown) to be inserted through talus 130. Also, generally “U-shaped” portion 905, coupled to locating tube 910 (i.e., a distal locator, provides a visual means of assessing the location of the fixation placement. This portion 905 and tube 910 also double as a clamp to secure guide assembly 900 and trial inserter assembly 700 in place during drilling. After the guide wire (not shown) has been installed, the surgeon may remove guide assembly 900 and trial inserter assembly 700. In other non-limiting embodiments, guide assembly 900 and trial inserter assembly 700 are left in place for drilling. Next, in step 1112, a hole is drilled in talus 130. The drill (not shown) is cannulated to coaxially align with the guide wire (not shown) and resist migration during drilling.

Once the hole is drilled, guide assembly 900 and trial inserter assembly 700 are removed and, in step 1114, an implant inserter 1005 is used to thread into implant device 110 as shown in FIG. 9C. Specifically, threaded rod 1010 is inserted through the talar tunnel (not shown) just formed from the drilling process and threaded into threaded hole 615 of implant device 110. Next, in step 1116, once implant device 110 is installed (shown in FIG. 6), the threaded rod 1010 is pulled back through the tunnel until implant device 110 comes into contact with talus 130. At this point, the surgeon can use a slap hammer (not shown) to engage holding spikes 600, 605 and 610 into talus 130.

As shown in FIG. 9D, after implant device 110 has been installed in place, in step 1118, implant inserter 1005 is removed and a dynamic fixation device 120 is placed on a screwdriver 1015 and inserted for ridged connection to implant device 110. Dynamic fixation device 120 comprises a first member positioned at a proximal end, a second threaded member positioned at a distal end and a tubular dynamic element coupled to first member and second threaded member. The tubular dynamic element cooperates with first and second members to exert a dynamic compressive force. The threaded dynamic element may be stretched, causing the first member and second threaded member to be in dynamic compression. In other non-limiting embodiments, any other type of screw device may be utilized. Next, in step 1120, as the dynamic fixation device 120 connects to implant device 110, the implant device 110 begins tensioning. Once a predetermined torque level has been reached, the spring element (not shown) contained within the dynamic fixation device 120 begins to stretch applying dynamic compression. This compression is maintained over a longer period of time as compared to normal static fixation devices. The method ends in step 1122.

It should be appreciated that FIGS. 9A-9D show the fixation of implant device 110 in the medial side of talus 130. However, in other non-limiting embodiments, a lateral approach may be utilized to install implant device 110 based on the location of neuro and vascular tissue located on the medial side. Thus, the present invention provides a hemiarthroplasty implant device 110 for talus resurfacing resulting in minimal removal of the underlying bone. Dynamic fixation device 120 allows for a short recovery as well as a lesser chance of implant loosening. Implant device 110 has a thickness that lowers stress shielding and includes medial and lateral components allowing for minimal removal of native tissue. Thus, implant device 110 provides a faster recovery period due to minimal invasive surgery that replaces just the damaged portion of the talar dome.

It should also be appreciated that implant device 110, in other non-limiting embodiments, may be utilized for tibial resurfacing. As such, the implant device 110 may vary in size in order to accommodate the articulating surface of the tibia. The implant device 110 may be selectively attached to the articulating surface of the tibial bone in order to replace any deteriorated or damaged bone or tissue.

Furthermore, in other non-limiting embodiments, a pair of implant devices 110 may be utilized concomitantly for tibia and talus resurfacing. Particularly, a pair of substantially similar implant devices 110 may be provided with varying sizes to accommodate the geometries of the talus and the tibia. The pair of implant devices 110 is selectively coupled to both the talus and the tibial bones in order to be concomitantly coupled to the articulating surfaces of the talar and tibial bones in a mirrored configuration. In this configuration, the tibial implant device 110 resides opposite the talar implant device 110. As such, the tibial implant device 110 may be shorter than the talar implant device 110 in order to accommodate the concomitant use of talar implant device 110 and tibial implant device 110. In this manner, the talus and tibial bones will be slidably coupled to each other by the plurality of implant devices 110. So, the tibial surface will have a mirrored configuration to the talar component.

It should be understood that this invention is not limited to the disclosed features and other similar method and system may be utilized without departing from the spirit and the scope of the present invention.

While the present invention has been described with reference to the preferred embodiment and alternative embodiments, which embodiments have been set forth in considerable detail for the purposes of making a complete disclosure of the invention, such embodiments are merely exemplary and are not intended to be limiting or represent an exhaustive enumeration of all aspects of the invention. The scope of the invention, therefore, shall be defined solely by the following claims. Further, it will be apparent to those of skill in the art that numerous changes may be made in such details without departing from the spirit and the principles of the invention. It should be appreciated that the present invention is capable of being embodied in other forms without departing from its essential characteristics.

1. A device for joint resurfacing, comprising:
a curved body for coupling to a talus bone, wherein said curved body further comprising a lateral portion and a medial portion, and further wherein said-curved body having an exterior surface for coupling to a tibial joint.

2. The device of claim 1, wherein said curved body further comprising a plurality of distinct radii to mimic a surface of said talus bone

3. The device of claim 2, wherein said plurality of distinct radii includes a medial convex portion and a lateral convex portion.

4. The device of claim 3, further comprising a plurality of spikes on an interior surface of said curved body.

5. The device of claim 4, wherein said plurality of spikes each have a tapered edge.

6. The device of claim 5, wherein said medial convex portion is coupled to said lateral convex portion at a midpoint of said curved body.

7. The device of claim 6, wherein said lateral convex portion is concave.

8. The device of claim 7, wherein said curved body further comprising a threaded hole disposed on said interior surface of said curved body.

9. The device of claim 7, wherein said threaded hole partially traverses said curved body

10. The device of claim 9, wherein said interior surface is provided for coupling to a talar dome.

11. The device of claim 10, wherein said plurality of holding spikes are tapered for traveling through bone material.

12. An assembly for joint resurfacing, comprising:
an implant device for attaching to a talus bone, wherein the implant device includes a curved body for coupling to a talus bone, wherein said curved body further comprising a lateral portion and a medial portion, and further wherein said curved body having an exterior surface for coupling to a tibial joint; and
a compression screw member for coupling to said implant device, wherein said compression screw member having a proximal screw member and a distal member.

13. The assembly of claim 12, wherein said curved body further comprising a plurality of distinct radii to mimic a surface of said talus bone

14. The assembly of claim 13, wherein said plurality of distinct radii includes a medial convex portion and a lateral convex portion.

15. The assembly of claim 14, further comprising a plurality of spikes on an interior surface of said curved body.

16. The assembly of claim 15, wherein said plurality of spikes each have a tapered edge.

17. The assembly of claim 16, wherein said medial convex portion is coupled to said lateral convex portion at a midpoint of said curved body.

18. The assembly of claim 17, wherein said lateral convex portion is concave.

19. The assembly of claim 18, wherein said curved body further comprising a threaded hole disposed on said interior surface of said curved body.

20. The assembly of claim 19, wherein said threaded hole partially traverses said curved body

21. The assembly of claim 20, wherein said interior surface is provided for coupling to a talar dome.

22. The assembly of claim 21, wherein said plurality of holding spikes are tapered for traveling through bone material.

23. The assembly of claim 22, wherein the compression screw member further comprises a proximal screw member having a first elongated body, wherein the first elongated body includes a first threaded portion at a first end and a bulbous portion at a second end.

24. The assembly of claim 23, wherein the bulbous portion includes a taper for providing an interference fit with said distal member.

25. The assembly of claim 24, wherein the taper provides for an interference lock with said distal member.

26. The assembly of claim 25, wherein the proximal screw member is cannulated having a circular cross-section with said first elongated body.

27. The assembly of claim 26, wherein the bulbous portion further includes an orifice longitudinally coextensive with a length of said bulbous portion.

28. The assembly of claim 27, wherein said orifice has a hexagonal shape, a star shape, or a square shape.

29. The assembly of claim 28, wherein said orifice is provided to receive a complementary shaped end of an instrument.

30. The assembly of claim 29, wherein said distal member further includes a second elongated body, wherein said second elongated body contains a second threaded portion at a third end, and an opening at a fourth end.

31. A method for bone resurfacing, comprising:
making a mid-foot incision in a foot;
distracting a tibial-talar joint;
inserting a guide wire into a-hole in a talus bone;
placing an implant device on the talus bone;
threading an implant inserter device into the hole and coupling to the implant device;
pulling the implant inserter device and abutting the implant device on a surface of the talus bone;
removing the implant inserter device and threadably coupling a dynamic fixation device to the implant device.

32. The method of claim 31, further comprising utilizing the trial inserter assembly to achieve a proper depth and alignment of the implant device.

33. The method of claim 32, wherein the implant further comprising a plurality of distinct radii to mimic a surface of the talus bone

34. The method of claim 33, wherein the plurality of distinct radii includes a medial convex portion and a lateral convex portion.

35. The method of claim 34, further comprising a plurality of spikes on an interior surface of the curved body.

36. The method of claim 35, wherein the plurality of spikes each have a tapered edge.

37. The method of claim 36, wherein the medial convex portion is coupled to the lateral convex portion at a midpoint of the curved body.

38. The method of claim 37, wherein the lateral convex portion is concave.

39. The method of claim 32, wherein the curved body includes a threaded hole disposed on the interior surface of the curved body.

40. The method of claim 39, wherein the threaded hole partially traverses the curved body

41. The method of claim 40, wherein the interior surface is provided for coupling to a talar dome.

42. The method of claim 41, wherein the plurality of holding spikes are tapered for traveling through bone material.
